(19) United States
(12) Patent Application Publication
(10) Pub. No.: US 2016/0010080 A1
(43) Pub. Date: Jan. 14, 2016

(54) A METHOD FOR PREPARING SILICA-COATED MAGNETIC BEAD

(71) Applicant: MagQu Co., LTD., New Taipei City (TW)

(72) Inventors: CHE-CHUAN YANG, New Taipei City (TW); CHIA-SHIN HO, New Taipei City (TW)

(73) Assignee: MagQu Co., LTD., New Taipei City (TW)

(21) Appl. No.: 14/327,542

(22) Filed: Jul. 9, 2014

(51) Int. Cl. C12N 15/10

(52) U.S. Cl. CPC C12N 15/1013

(57) ABSTRACT

The present invention relates to a method of preparing a sedimentation adjustment silica-coated magnetic bead, comprising the following steps: (1) preparing a magnetic core; (2) adding various ratio of silica coating material to coat the magnetic core with silica forming a silica-coated magnetic bead; and (3) washing the silica-coated magnetic bead sequentially by an alcoholic solvent and water. The present invention also relates to a silica-coated magnetic bead prepared by the above method, comprising a magnetic core and a tunable silica layer, wherein the thickness of the tunable silica layer is ranging from 0.77-2.31 μm.

### FIELD OF THE INVENTION

The present invention relates to a method for preparing silica-coated magnetic bead.

### BACKGROUND OF THE INVENTION

Bio-functionalized magnetic particles have been applied to biomedicines. Various bio-applications require different sizes of magnetic particles. For example, due to the strong magnetism of each particle, magnetic particles with micrometers in diameter are useful for in-vitro extraction or purification of bio-molecules like antibodies, proteins, and nucleic acids.

Recently small nucleotide plays important roles in different biological pathway, which causes the need to provide silica-coated magnetic beads to capture these important small fragments of nucleotide.

Thus, there is a demand for improved silica-coated particles for binding to small fragments of DNA, especially small nucleotide fragments under 100 base pairs. Further, there is a demand for silica-coated particles having thickness-tunable silica layer capable of being customized for different applications in biomedicine.

### SUMMARY OF THE INVENTION

The present invention relates to a method of preparing a sedimentation adjustment silica-coated magnetic bead, comprising the following steps: (1) preparing a magnetic core; (2) adding various ratio of silica coating material to coat the magnetic core with silica forming a silica-coated magnetic bead; and (3) washing the silica-coated magnetic beads sequentially by alcoholic solvent and water.

The present invention also relates to a silica-coated magnetic bead prepared by the above method of the present invention, comprising a magnetic core and a tunable silica layer, wherein the thickness of the tunable silica layer is ranging from 0.77-2.31 μm.

### BRIEF DESCRIPTION OF THE DRAWINGS

FIG. 1 illustrates a flow chart of preparing a sedimentation adjustment silica-coated magnetic bead of the present invention.

FIG. 2 illustrates the observation results of the sedimentation of silica-coated magnetic beads in solution at various time periods (0, 10, 90 and 120 seconds).

FIG. 3 illustrates small fragments being captured by the silica-coated magnetic beads of the present invention, as compared to other brands (Brand A and Brand B). (A) illustrates the observation result of the small fragments being captured by the silica-coated magnetic beads of the present invention and other brands (Brand A and Brand B). (B) illustrates the electrophoresis result of the small fragments being captured by the silica-coated magnetic beads of the present invention and other brands (Brand A and Brand B).

### DETAILED DESCRIPTION OF THE INVENTION

The present invention is more fully appreciated by reference to the following description, including the following glossary of terms and the concluding examples. For the sake of brevity, the disclosures of the publications, including patents, cited in this specification are herein incorporated by reference.

As used herein in the specification, “a” or “an” may mean one or more. As used herein in the claim(s), when used in conjunction with the word “comprising”, the words “a” or “an” may mean one or more than one.

The present invention provides a method of preparing a sedimentation adjustment silica-coated magnetic bead, comprising the following steps: (1) preparing a magnetic core; (2) adding various ratio of silica coating material to coat the magnetic core with silica forming a silica-coated magnetic bead; and (3) washing the silica-coated magnetic bead sequentially by an alcoholic solvent and water.

In one embodiment, the step (1) of the above method further comprises the following steps: (a) mixing a ferric salt and a ferrous salt in an alkaline solution and heating from 60° C. to 90° C. to form the magnetic core; (b) cooling the magnetic core to room temperature; and (c) adding the alkaline solution and the alcoholic solvent to the magnetic core.

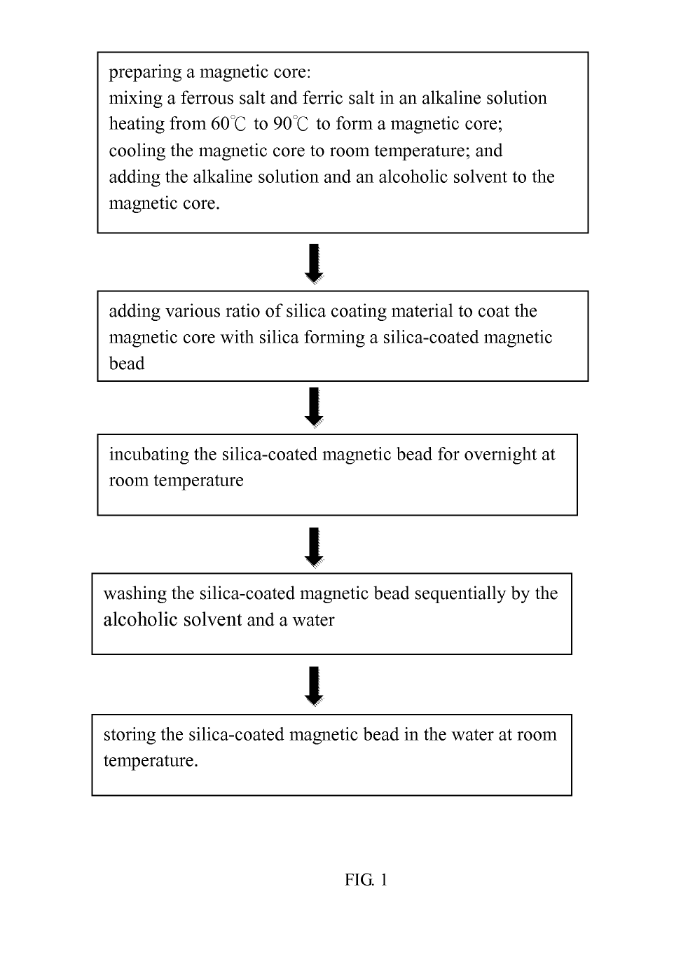

FIG. 1 illustrates the complete procedure of preparing a sedimentation-adjusted silica-coated magnetic bead: (i) preparing a magnetic core; (ii) adding various ratio of silica coating material to coat the magnetic core with silica forming a silica-coated magnetic bead; (iii) incubating the silica-coated magnetic bead for overnight at room temperature; (iv) washing the silica-coated magnetic bead sequentially by an alcoholic solvent and a water; and (v) storing the silica-coated magnetic bead in the water at room temperature.

As used herein, “ferric salt” refers to a ferric iron-containing-compound or a material, also denoted Fe(III) salt. In one preferred embodiment, the ferric salt is FeCl₃. As used herein, “ferrous salt” refers to a ferrous iron-containing-compound or a material, also denoted Fe(II) salt. In one preferred embodiment, the ferrous salt is FeCl₂.

According to an example embodiment, the ferric salt is 0.02 to 0.42 mole and the ferrous salt is 0.024 to 0.3 mole.

In one embodiment, the alkaline solution is 5 to 25%. In one preferred embodiment, the pH value of the alkaline solution is more than 11.

According to another example embodiment, the added volume of the alkaline solution in the step (c) is 0.05 to 0.1 times based on a mixture volume of the step (a). In another embodiment, the added volume of the alcoholic solvent in step (c) is 1 to 3 times based on a mixture volume of the step (a).

In one embodiment, the alcoholic solvent includes but is not limited to a solvent containing an organic compound in which the hydroxyl functional group (—OH) is bound to a carbon atom. In a preferred embodiment, the alcoholic solvent is ethanol. In another embodiment, the water is ddH₂O.

According to a further example embodiment, the various ratio of silica coating material is 0.05% to 2%. In another embodiment, the step (2) of the method of preparing a sedimentation-adjusted silica-coated magnetic bead controls the sedimentation rate of the silica-coated magnetic bead.

The term “silica coating material” used herein means a crosslinking agent in silicone polymers and as a precursor to silicon dioxide, including but not limited to a tetrabutyl orthosilicate, a tetrapropoxysilane, a tetraethyl orthosilicate, a tetraisopropyl orthosilicate or a sodium silicate.

The invention also provides a silica-coated magnetic bead prepared by the method of preparing a sedimentation-adjusted silica-coated magnetic bead of the present invention, comprising a magnetic core and a tunable silica layer, wherein the thickness of the tunable silica layer is ranging from 0.77-2.31 μm.

In one embodiment, the thickness of the tunable silica layer is ranging from 0.65-1.9 μm. In a preferred embodiment, the thickness of the tunable silica layer is ranging from 0.42-1.54 μm.

In one embodiment, the material of the tunable silica layer is a tetrabutyl orthosilicate, a tetrapropoxysilane, a tetraethyl orthosilicate, a tetraisopropyl orthosilicate or a sodium silicate.

According to an example embodiment, the silica-coated magnetic bead further is bound to a small nucleotide. In a preferred embodiment, the length of the small nucleotide is 10˜100 base pair. In a more preferred embodiment, the small nucleotide is RNA or DNA.

### EXAMPLES

The examples below are non-limiting and are merely representative of various aspects and features of the present invention.

### Example I

### Preparing Silica-Coated Magnetic Beads

Procedure of preparing silica-coated magnetic beads of the present invention:
1. 0.02˜0.42 mole of FeCl₃ and 0.02˜0.3 mole of FeCl₂ in 18˜1500 ml water was heated at 60˜90° C. for 10˜30 minutes to obtain a mixture.
2. The above mixture was mixed with 5˜25% of alkaline solution and stirred at 60˜90° C. for 20˜45 minutes to form magnetic cores.
3. Cooled the magnetic cores to room temperature.
4. Alkaline solution (0.05X˜0.1X volumes) and ethanol (1X˜3 X volumes) were then mixed to step 3.
5. Tetraethyl orthosilicate (TEOS) was added to step 4 in various ratio (0.05%˜2%) to coat the magnetic cores with silica forming silica-coated magnetic beads.
6. The silica-coated magnetic beds were incubated overnight at room temperature.
7. The silica-coated magnetic beads were washed sequentially by ethanol and ddH₂O.
8. The silica-coated magnetic beads were finally stored in ddH₂O at room temperature.

### Example II

### Particle Size Analysis of Silica-Coated Magnetic Beads

After obtaining the silica-coated magnetic beads, the particle size of the silica-coated magnetic beads of the present invention were further analyzed. The analysis steps included the following:
1. Turned on the analyzer “HORIBA LA-300” and software.
2. Warmed up for 30 minutes.
3. Added 300 ml ddH₂O into the analyzing chamber and turned on the circulation system.
4. Checked the parameter and ran blank.
5. 100 μl of each silica-coated magnetic beads were added into analyzing chamber separately and analyzed on sonication mode for 20 minutes.
6. The diameter was recorded.

Table 1 shows the mean diameters of series of the silica-coated magnetic beads of the present invention.

TABLE 1
The mean diameters of series of the
silica-coated magnetic beads

| Bead series | Mean Diameter (μm)* |
|---|---|
| Type 1 | 2.93 |
| Type 2 | 3.35 |
| Type 3 | 3.94 |
| Type 4 | 4.15 |
| Type 5 | 4.47 |

*The diameter was analyzed by DLS (LA-300; HORIBA)

The different particle sizes of above series of silica-coated magnetic beads came from the various thicknesses of the tunable silica layer. The thickness of the tunable silica layer is calculated by the following formula: (the mean diameter of Type X)−(the mean diameter of Type Y). For example, the difference in thickness of the tunable silica layers between type 5 and type 1 is 1.54 μm (4.47−2.93=1.54). Therefore, the range of the thickness of the tunable silica layer was from 0.42-1.54 μm.

### Example III

### Sedimentation of Silica-Coated Magnetic Beads in a Solution

The analyzing steps of the sedimentation rate of the silica-coated magnetic beads:
1. 10 mg of each silica-coated magnetic beads were taken and re-suspended in 200 ml of ddH₂O.
2. Transferred the beads to glass tubes.
3. Vortexed the glass tubes vigorously for 20 sec.
4. Fixed and stood the tubes vertically.
5. The sedimentation of beads in ddH₂O was observed and recorded by photos at various time periods.

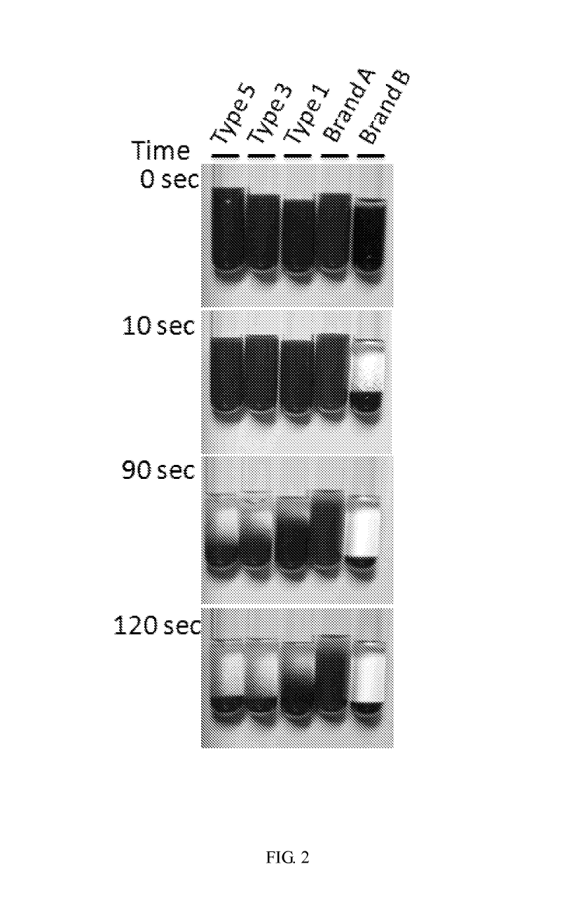

FIG. 2 illustrates the time-period observations of sedimentation of silica-coated magnetic beads in the solution. The silica-coated magnetic beads of the present invention (Types 1, 3 and 5), Brand A and Brand B were suspended in ddH₂O. The sedimentation was recorded at various time periods (0, 10, 90 and 120 seconds). The silica-coated magnetic beads of the present invention showed tunable sedimentation characteristics that other brands of magnetic silica beads did not have.

Table 2 further shows the result of the sedimentation rate of those five silica-coated magnetic beads illustrated on FIG. 2. Type 1 and Brand B showed quicker sedimentation rate. Brand A showed the slowest sedimentation rate.

TABLE 2
Sedimentation character of silica-coated magnetic beads

| Beads | Type 5 | Type 3 | Type 1 | Brand A | Brand B |
|---|---|---|---|---|---|
| Sedimentation | ++ | +++ | ++++ | + | +++++ |

The sedimentation character of testing magnetic silica beads was distinguished in levels.
More “+” means quicker sedimentation.

### Example IV

### Capturing Ability of Small Fragments (<100 bp) of DNA

The procedure of preparing silica-coated magnetic beads binding to DNA fragments:
1. Took 1 mg of silica-coated magnetic beads which made by the present invention, Brand A and Brand B to eppendorf separately.
2. Put the eppendorf on a magnetic stand (Magdorf, purchased from MagQu) for 10 sec then removed the supernatant by aspiration.
3. Added 100 μl of ddH₂O to each eppendorf, then removed the eppendorf from the magnetic stand and vortexed for 5 sec.
4. Put the eppendorf on the magnetic stand (Magdorf, purchased from MagQu) for 10 sec then removed the supernatant by aspiration.
5. Repeated step 4-5 twice.
6. 100 μl of DNA Binding Buffer with 5 mg of 20 by DNA ladder was mixed with each silica-coated magnetic beads in the eppendorf and then incubated by slightly vibration for 2 min at room temperature.
7. Put the eppendorf on a magnetic stand (Magdorf, purchased from MagQu) for 10 sec then removed the supernatant by aspiration.
8. Added 100 μl of Wash Buffer to each eppendorf, then removed the eppendorf from the magnetic stand and vortexed for 5 sec.
9. Put the eppendorf on the magnetic stand (Magdorf, purchased from MagQu) for 10 sec then removed the Wash Buffer by aspiration.
10. Kept the tap of eppendorf open, and removed them to an oven for 10 min to dry the silica-coated magnetic beads.
11. 20 μl Elution Buffer were added.
12. Pippetted the beads for 10 times.
13. Put the eppendorf on the magnetic stand (Magdorf, purchased from MagQu) for 10 sec.
14. Collected the eluted supernatant to a new eppendorf and added 4 μl of 6× DNA loading dye to each sample.
15. 3% of agarose gel was used to separate the eluted DNA by electrophoresis.
16. The DNA was visualized by EtBr staining.

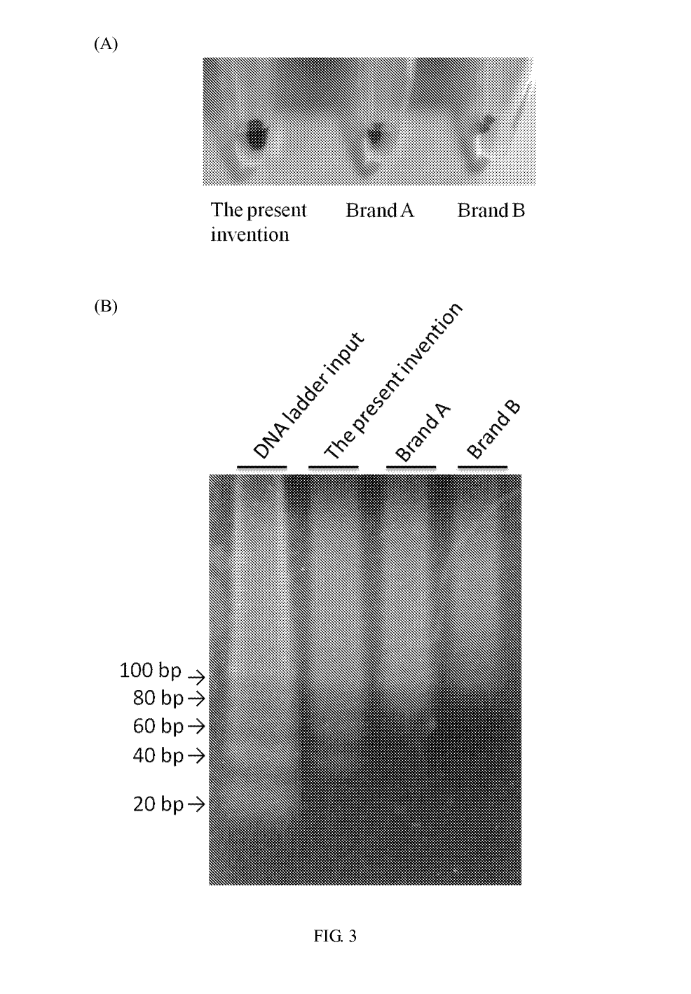

FIG. 3 illustrates small fragments being captured by the silica-coated magnetic beads of the present invention, as compared to other brands (Brand A and Brand B). FIG. 3 (A) shows the observation result of the small fragments being captured by the silica-coated magnetic beads of the present invention and other brands (Brand A and Brand B). The silica-coated magnetic beads of the present invention in FIG. 3 (B) show higher efficacy of capturing small fragments of DNA (100-40 bp) than other brands.

Those skilled in the art recognize the foregoing outline as a description of the method for communicating hosted application information. The skilled artisan will recognize that these are illustrative only and that many equivalents are possible.

What is claimed is:

1. A method of preparing a sedimentation adjustment silica-coated magnetic bead, comprising the following steps:
(1) preparing a magnetic core;
(2) adding various ratio of silica coating material to coat the magnetic core with silica forming a silica-coated magnetic bead; and
(3) washing the silica-coated magnetic bead sequentially by an alcoholic solvent and water.

2. The method of claim 1, wherein the step (1) further comprising the following steps:
(a) mixing a ferric salt and a ferrous salt in an alkaline solution and heating from 60° C. to 90° C. to form the magnetic core;
(b) cooling the magnetic core to room temperature; and
(c) adding the alkaline solution and the alcoholic solvent to the magnetic core.

3. The method of claim 2, wherein the ferric salt is 0.02 to 0.42 mole and the ferrous salt is 0.024 to 0.3 mole.

4. The method of claim 2, wherein the alkaline solution is 5 to 25%.

5. The method of claim 1, wherein the various ratio of silica coating material is 0.05% to 2%.

6. The method of claim 1, wherein the step (2) controls the sedimentation rate of the silica-coated magnetic bead.

7. The method of claim 1, wherein the silica coating materials is a tetrabutyl orthosilicate, a tetrapropoxysilane, a tetraethyl orthosilicate, a tetraisopropyl orthosilicate or a sodium silicate.

8. A silica-coated magnetic bead prepared by the method of claim 1, comprising a magnetic core and a tunable silica layer, wherein the thickness of the tunable silica layer is ranging from 0.77-2.31 μm.

9. The silica-coated magnetic bead of claim 8, wherein the silica-coated magnetic bead further is bound to a small nucleotide.

10. The silica-coated magnetic bead of claim 9, wherein the length of the small nucleotide is 10˜100 base pair.

11. The silica-coated magnetic bead of claim 9, wherein the small nucleotide is RNA or DNA.
